(19) United States
(12) Patent Application Publication
(10) Pub. No.: US 2025/0208151 A1
(43) Pub. Date: Jun. 26, 2025

(54) KIT OF PARTS AND MICROBIOLOGICAL METHOD FOR ASSESSMENT OF THE FOLATE STATUS IN SERUM AND RED BLOOD CELLS

(71) Applicant: Immundiagnostik AG, Bensheim (DE)

(72) Inventors: Franz Paul Armbruster, Bobenheim-Roxheim (DE); Hendrik Seidl, Bensheim (DE)

(73) Assignee: Immundiagnostik AG, Bensheim (DE)

(21) Appl. No.: 18/848,851

(22) PCT Filed: Mar. 11, 2023
(86) PCT No.: PCT/EP2023/056257

§ 371 Date: Sep. 19, 2024

(30) Foreign Application Priority Data
Mar. 21, 2022 (DE) 10 2022 106 594.4

(51) Int. Cl. G01N 33/82

(52) U.S. Cl. CPC G01N 33/82; G01N 2333/335; G01N 2333/948

(57) ABSTRACT

Kit and method for the microbiological determination of folate and folic acid in a whole blood sample and for the assessment of the folate status of an individual, comprising steps for complete lysis of erythrocytes (red blood cells) and release of folate species, as well as steps for release of lysosomal γ-glutamyl hydrolase from cells contained in the whole blood sample and/or addition of γ-glutamyl hydrolase and complete enzymatic hydrolysis of the γ-glutamyl chains of folypolyglutamate species, folytetraglutamates, folypentaglutamates and folyhexaglutamate, followed by a microbiological assay for comparative study of growth and metabolism in the absence and presence of various amounts of treated whole blood sample and/or folate calibrator to assess the folate status of an individual in comparison to the folate references.

### FIELD OF THE INVENTION

The present invention concerns a microbiological method for assessing the folate status of a person (C12Q 1/02).

### TECHNICAL BACKGROUND

Vitamins are substances that the human organism must uptake with food, and therefore specific deficiencies can occur in case of disease, malnutrition, or malabsorption. The B vitamins are the class of water-soluble vitamins, and though these vitamins share similar names (B1, B2, B3, etc.), they are chemically distinct. The B vitamins are referred to by each specific number, such as B1 for thiamine, B2 for riboflavin, and B3 for niacin. Some B vitamins are more commonly recognized by name than by number niacin, pantothenic acid, biotin, and folate. Folate occurs naturally in leafy vegetables, where it received its name, and folic acid is the synthetic form of this vitamin. The vitamin activity of naturally occurring folate is less than the one of folic acid because not all naturally occurring folate compounds have vitamin activity. Folate is essential for cell growth and replication. Inadequate folate uptake is associated with adverse health outcomes, and a low maternal folate status can be associated with pre-eclampsia, spontaneous abortion, stillbirth, preterm delivery, low birth weight, autism, neural tube defects, and various congenital anomalies of the spine and brain (see for review WHO's Vitamin and Mineral Nutrition Information System—VMNIS 7, G 15.01 of 2015)

Although folate is mainly stored in the liver, the folate status of a person can be assessed in serum, plasma, red blood cells, and urine. The serum folate level is an indicator of recent folate intake only. Therefore, a single folate measurement in serum cannot be used to differentiate between a transitory decrease in dietary folate intake and a chronic deficiency. Conversely, red blood cell folate concentrations respond slowly to changes in folate intake as the erythrocytes, which have a 120-day lifespan, accumulate folate during erythropoiesis. Therefore, the folate content of a whole blood sample could be used for assessing the long-term folate status of a person.

The folate status of an individual is further dependent on numerous factors, including age, pregnancy, lactation, socioeconomic status and access to dietary folate, and co-existing physiological factors such as the levels of homocysteine and other vitamins. Genetics must also be considered, particularly the polymorphisms in the methylenetetrahydrofolate reductase gene and mutations in the human glutamate carboxypeptidase gene II. As mentioned in the WHO report, pregnant women with low folate status are at increased risk of bearing children with neural tube defects and congenital heart defects. People with low folate status are generally at increased risk for metabolic disorders, cardiovascular disease, colon cancer, altered cognition, particularly in the elderly, including Alzheimer's disease (US 2004/0185487).

The folate concentration in a sample can be determined (i) by chemical-physical methods, for example, by high-pressure liquid chromatography coupled to various detectors such as mass spectrometry, (ii) by immunological methods, (iii) by animal experiments which are not relevant in practice, and (iv) by microbiological methods. U.S. Pat. No. 8,663,946 B2 (Fu Tzu-Fun) discloses a method and kit for detecting one or more folates which comprises the steps: (a) mixing a sample and an extraction buffer to form a mixture, boiling and then cooling the mixture to ambient temperature, and separating a supernatant from the mixture by centrifugation; (b) adding a recombinant γ-glutamyl hydrolase (GGH) and a folate conversion enzyme to the supernatant to drive a reaction; (c) stopping the reaction, and (d) analyzing the reaction mixture by high-performance liquid chromatography to quantify the one or more folates. Other chemical and chromatographic methods for detecting folate have been disclosed by Doherty R F, Beecher R A in A method for analysis of natural and synthetic folate in foods, J Agricultural and Food Chemistry, 2003, p. 354-361; Chao Wang et al. in A liquid chromatography-tandem mass spectrometric method for the quantitative determination of native 5-methyltetrahydrofolate and its polyglutamyl derivatives in raw vegetables, J Chromatography B, 2010, 2949-2958. EP1472545 (Axis-Shield ASA) describes a method of assaying for folate, subjecting the sample to hydrolysis to release para-aminobenzoic acid para-aminobenzoyl glutamic acid, or a salt thereof; contacting the released compounds with a diazo binding partner and directly or indirectly detecting the resulting binding partner. U.S. Pat. No. 6,329,162 assigned to AntiCancer Inc. patents a method for assessing the level of folate in a biological sample which comprises:—providing said sample with glycine N-methyltransferase (GMT) and with an excess of S-adenosyl methionine (SAM) and of glycine; providing a control which contains no folate with said GMT and excess SAM and glycine in comparable amounts to those supplied to the sample; and comparing the concentration of at least one product formed in the sample with the concentration of said product formed in control, whereby the difference in levels of said product in the sample as compared to the control is directly proportional to the level of folate in the sample

The chemical-physical and immunological methods are generally not very sensitive and helpful because folate is a trace vitamin and because of the many biologically active inactive folate species. The microbiological methods for assessing the folate status developed in the 1960s still form the basis for current methods since genetically modified strains of Lactobacillus rhamnosus have been made available for the determination of. This Lactobacillus strain is responsive to multiple forms of folate while excluding those species without vitamin activity. However, a recent study compared the results of microbiological assays from three laboratories, and it was observed that substantially different results were obtained for the folate status of an individual. Concerning the technical difficulties, microbiological assays typically require one or more dilution series of the sample in assay buffer so that the growth or turnover value of the test microorganism falls within the measurement range of the parallel standard concentration series at the end of the incubation period. A standard curve must be generated for each assay. In addition, for safety and accuracy reasons, each concentration level of the standard and the sample series must be used at least three times. The folate content of the sample is determined by comparison with the known folate content of the parallel standard series. Generalized precision data are not possible; however, the coefficient of variation should be about 10 percent or less. Moreover, the inoculum for the standard and sample series must be added correctly and have the desired sensitivity and specificity, which is sometimes uncertain. For further information, please confer. —Gorin G et al. in Determination of niacin . . . with lyophilized Lactobacillus arabinosus ATCC 8014, Appl Microbiol 1970, 20, 641-642; Conrad P B et al. in Stabilization and preservation of Lactobacillus acidophilus in saccharde matrices, Cryobiology 2000, 41, 17-24; Kelleher B P et al. in Microbiological assay for vitamin B12 performed in 96-well microtitre plates, J Clin Pathol 1991, 44(7), 592-595; Bui M H in A microbiological assay on microtitre plates of thiamine in biological fluids and foods, J Vitam Nutr Res 1999, 69(5), 362-366.

Molloy A M et al. in Microbiological assay for serum, plasma, and red cell folate using cryopreserved microtiter plate method, Methods Enzymol 1997, 281, 43-53 teach that despite microbiological assays are difficult to set up and challenging to maintain, they remain the “gold standard” and the method of choice. However, the routine analysis of serum and erythrocyte folate remained still the subject of controversy, and only a few laboratories in the food sector are specialized in this type of analysis, and even fewer clinical laboratories in human diagnostics. The microbiological method is labor-intensive and requires considerable laboratory organization, but the technique has been refined and automated to such an extent that it is easy to perform, reliable to maintain, and considerably less costly than alternative methods, mainly where large numbers of samples are involved. Three crucial advances in assay technology have contributed to this—the development of genetically modified strains of organisms and Lactobacillus rhamnosus resistant to antibiotics. Second, cryopreservation of the inoculum in multiple individual vials results in standardized growth curves that are reproducible for hundreds of assays. Last, automated microtiter plate technology and its associated computerized analysis packages developed for enzyme-linked immunosorbent assays (ELISAs) are suited to measuring the turbidity of microbiological growth. Although microbiological methods for determining the active folate content in a human biological sample are available, the assessment of the—long-term—folate status of an individual remains a problem and is prone to errors. State of the art represents a problem.

### BRIEF DESCRIPTION OF THE INVENTION

This problem is solved by a method for microbiological determination of folate and folic acid in a whole blood sample and assessment of the folate status of an individual, comprising the steps of:
preparing one or more culture vessels for microbiological growth and metabolism determination that contain a pre-determined number of vital cells of Lactobacillus rhamnosus;
   obtaining a defined sample amount of whole blood from an individual whose folate status is to be determined;
adding to the sample of whole blood a predetermined amount of red blood cell lysis buffer to obtain lysis of red blood cells for a release of folate species;
adding an amount of γ-glutamyl hydrolase and/or a surfactant capable of permeabilization of lysosomes for a release of lysosomal γ-glutamyl hydrolase from cells contained in the whole blood sample;
treating and incubating the lysed blood sample at pH 5.5 to 7 for some time to obtain enzymatic hydrolysis of the γ-glutamyl chains of folypolyglutamate species, folytetraglutamates, folypentaglutamates, and folyhexaglutamate;
performing a microbiological assay for comparative examination of growth and metabolism in the absence and presence of differing amounts of treated whole blood sample and/or folate calibrator to assess the individual's folate status compared to the folate references.

In some embodiments, the red blood cell lysis buffer contains 1% ascorbate/ascorbic acid, pH 4.2 to 5.0, and a detergent for permeabilization of lysosomes.

In some embodiments, the surfactant for permeabilization of lysosomes is selected from sapogenins, steroidal sapogenins, saponins, triterpene glycosides, terpenoids, alkylphenol-ethoxylates, (Triton® X-100), nonylphenol-ethoxylates, octylphenol-ethoxylates.

In some preferred embodiments of the invention, the enzymatic hydrolysis of the γ-glutamyl chains of folypolyglutamate species is done in a phosphate buffer of pH 5.5 to 6.5 at ambient temperature to 37 degrees Celsius for 10 to 30 minutes, if not done in an ascorbic/ascorbate buffer at pH 4.2.

In some embodiments, the Lactobacillus rhamnosus is grown in an assay medium buffered at pH 6 to offset the inhibitory effects of produced lactic acid.

In some preferred embodiments of the invention, the Lactobacillus is Chloramphenicol-resistant Lactobacillus rhamnosus ATCC 7469.

In some most preferred embodiments, the microbiological assay in a microtiter plate.

Another aspect of the invention is a test kit for assessment of the folate status of an individual by a microbiological assay of folate and/or folic acid in a whole blood sample, comprising:
a microtiter plate for the microbiological growth and metabolism determination, which cavities each contain a predetermined number of vital cells of Lactobacillus rhamnosus, that have been rendered durable for dry storage at ambient temperature by shock-freezing and freeze-drying;
an ascorbic acid/ascorbate buffer system, pH 4.0 to 4.5

Preferred embodiments of the invention can be taken from the detailed description, and the example described below.

### BRIEF DESCRIPTION OF THE DRAWING

In the drawings:

FIG. 1 is a graphical representation (standard calibration curve) of the growth of Lactobacillus rhamnosus within the microtiter wells in dependence of the folate concentration (OD measured at 630 nm—measurements in duplicates)

### A DETAILED DESCRIPTION OF THE INVENTION

The folates, also referred to as vitamin B9, differ in oxidation state, carbon substitution, and glutamate residues. Folic acid does not occur naturally and is present in individuals only who take vitamin supplements or eat fortified foods. Reduced folates are less stable than folic acid, and stabilities depend on the one-carbon substitution. Oxidation usually results in folic compounds lacking vitamin activity, although some may be converted to biologically active oxidized forms. The high number of folate derivatives, the instability of some, and the potential of some of them to interconvert chemically complicate the assessment of the vitamin B9 or folate status. Assays based on competitive protein binding have become popular because of their availability in commercial kit form. Some laboratories have also introduced mass spectrometry methods to measure individual folate one-carbon forms. Human plasma and serum naturally contain folate monoglutamates, mostly the 5-methyltetrahydrofolate form (5-methyl-THF), whereas erythrocytes contain mainly polyglutamates of 5-methyl-THF. The assessment of the folate status in whole blood samples is challenging because pteroyl polyglutamates cannot be metabolized by Lactobacillus rhamnosus. In contrast, such a biological sample would be suitable for assessing the long-term folate status of an individual. There is also an increased interest in assaying the folate one-carbon forms because the above-mentioned genetic polymorphisms also cause a redistribution of the folate forms between serum and red blood cells. In addition, the use of folate supplements has resulted in the appearance of free folic acid in blood samples. The excessive presence of synthetic forms of folate may have detrimental effects on an individual's health, not just impact the assessment of their folate status.

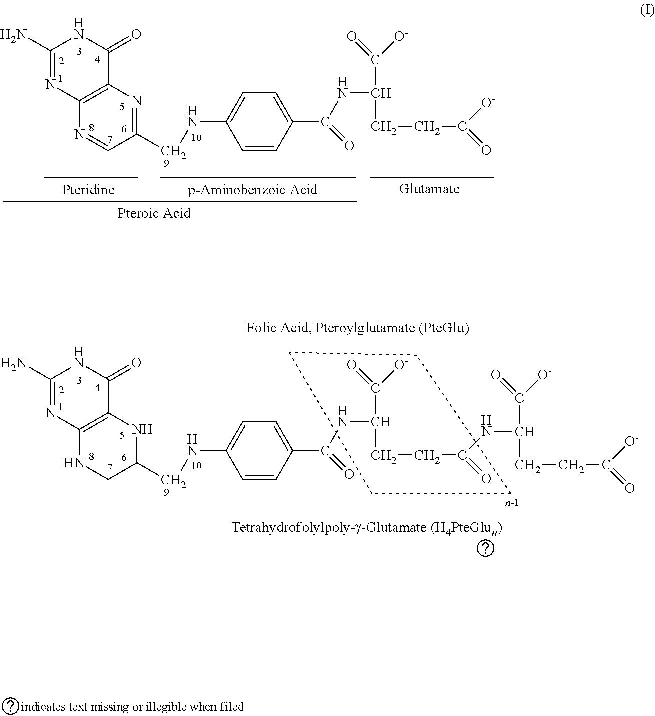 [SMILES: Nc1nc2ncc(CNc3ccc(C(=O)NC(CCC(=O)[O-])C(=O)[O-])cc3)nc2c(=O)[nH]1 ; Nc1nc2c(c(=O)[nH]1)NC(CNc1ccc(C(=O)NC(CCC(=O)NC(CCC(=O)[O-])C(=O)[O-])C(=O)[O-])cc1)CN2]

On the other hand, folate is essential for normal cell growth and replication, and folate and vitamin B₁₂ deficiencies have been acknowledged as the most common causes of macrocytic anemia (Kaferle J, Strzoda C E in Evaluation of macrocytosis, Am Fam Physician. 2009, 79(3):203-8). Most importantly, a poor maternal folate status can be linked to abruptio placentae, pre-eclampsia, spontaneous abortion, stillbirth, preterm delivery, low birth weight, and severe congenital anomalies of the brain and spine, such as neural tube defects (NTDs) (Molloy A M et al. in Effects of folate and vitamin B₁₂ deficiencies during pregnancy on fetal, infant, and child development, Food Nutr Bull 2008; 29(2):101-115; Hibbard B M et al. in Folic acid and reproduction, Acta Obstet Gynecol Scand. 1965, 44(3):375-400).

Microbiological assays using Lactobacillus rhamnosus (also known as Lactobacillus casei) are recommended and used for determining the folate content in foods as they are responsive to multiple forms of folate, excluding only those without vitamin activity (Anderson B B et al. in Effect of light on the Lactobacillus casei microbiological assay, Am J Clin Pathol 1968, 21:85-7; Yetley E A et al. in Biomarkers of folate status in NHANES: a roundtable summary, Am J Clin Nutr. 2011; 94(1):303S-312S. doi:10.3945/ajcn.111.013011). On the other hand, a recent study comparing microbiological assays from three laboratories showed that the different results were obtained because of unclear problems, not just with the species of the folate calibrator but also with the genetics of the microorganism (Pfeiffer C M et al. in Comparison of serum and red blood cell folate microbiologic assays for national population surveys, J Nutr. 2011, 141(7):1402-9. doi:10.3945/jn.111.141515). The present inventors found that the different results were likely due to the under-recovery of 5-methyltetrahydrofolate because the higher polyglutamate folate species in red blood cells were on the one hand not degraded to usable folate forms having vitamin activity, and on the other hand got precipitated during cell lysis or entrapped with the cell membranes or because the γ-glutamyl hydrolase endogenous of whole blood was not active or released from the lysosomes. The invention solves these problems and provides a kit and assay protocol that ensures a complete release and solubilization of all folate species and an optimized enzymatic treatment of the pteroyl-polyglutamates by a γ-glutamyl hydrolase that is set free in the reaction mixture by specific permeabilization of lysosomes from blood cells. The method of the invention is particularly useful when assessing the folate status in ranges generally qualified as low and too low. The removal of the potential under-recovery of 5-methyl-THF greatly contributes to a correct assessment of an individual's folate status. It increases the reliability of the clinical diagnostics and improves the medication with folate and vitamin supplements, particularly for pregnant women.

The lysis of erythrocytes is conventionally done by the action of 1% ascorbic acid, typically unbuffered, but while ascorbic acid is known as a potent reducing agent which can interfere with many blood chemical tests, it was not known that its pKa of 4.17 and 11.6 also leads to partial precipitation and entrapment of the higher water-soluble vitamin B9s in erythrocytes so that they could not be degraded to pteroyl-monoglutamates. Thus, this proportion of vitamin B9 or active folate was removed from the microbiological assays for folate and led to an under-recovery of 5-methyl-THF. These problems are overcome by using a buffered system of ascorbic acid and ascorbate at pH 4.2 for cell lysis of whole blood samples and by adding a surfactant to the whole blood sample, making lysosomes permeable to the lysosomal γ-glutamate hydrolase.

### EXAMPLES

### Example 1 Hydrolysis and Release of Pteroyl Polyglutamate from Erythrocytes

Theory. Folic acid (N-(4-{[(2-amino-4-oxo-1,4-dihydropteridin-6-yl)methyl]-amino}-benzoyl)-L-glutamic acid or pteroyl-L-glutamic acid) is present in erythrocytes (red blood cells) not as pteroyl-L-glutamic acid (monomer) but as polyglutamate (pteroyl-polyglutamate); see formulae I above. The WHO recommends the measurement of folate levels in whole blood by a microbiological assay based on genetically modified Lactobacillus rhamnosus ATCC 7469, which measures only short pteroyl-polyglutamates with less than three glutamates. The inventors identified a need to maintain higher molecular weight pteroyl-polyglutamates in solution during cell lysis and for hydrolysis and degradation by lysosomal γ-glutamase (glutamate conjugase) to 5-methyl-THF for correct physiological assessment of an individual's folate status (vitamin B9 status). The complete release and solubilization of higher pteroyl-polyglutamates from red blood cells is particularly critical when an individual is likely to be suffering from inadequate folate uptake, as the precipitated or entrapped pteroyl-polyglutamates may then constitute 30 to 50 percent of the total erythrocyte folate when cell lysis is induced by an unbuffered 1% ascorbic acid solution (pH 2.5) as recommended in the prior art.

Test solutions and buffers: The 96-well plate was prepared as described in European Patent No. 1 774 021 B1, examples 1 and 2. The assay medium (ASYMED) was Difco™ Folic Acid Casein Medium containing activated charcoal-treated pancreas digested casein 10.0 g/L, dextrose 40.0 g/L, sodium acetate 40.0 g/L, potassium dihydrogen phosphate 1.0 g/L, dipotassium hydrogen phosphate 1.0 g/L, DL-tryptophan 0.2 g/L, L-asparagine 0.6 g/L, L-cysteine hydrochloride 0.5 g/L, adenine sulfate 10.0 mg/L, guanine hydrochloride 10.0 mg/L, uracil 10.0 mg/L, xanthine 20.0 mg/L, polysorbate-80 0.1 g/L, glutathione (reduced) 5.0 mg/L, magnesium sulfate 0.2 g/L, sodium chloride 20.0 mg/L, iron sulfate 20.0 mg/L, manganese sulfate, 15.0 mg/L, riboflavin 1.0 mg/L, p-aminobenzoic acid 2.0 mg/L, pyridoxine hydrochloride 4.0 mg/L, thiamine hydrochloride 400.0 μg/L, calcium pantothenate 800 μg/L, nicotinic acid 800 μg/L, biotin 20 μg/L, 0.05% ascorbic acid. The storage medium also contained 200 mmol/L trehalose, 10 mmol/L CaCl₂. The folic acid standard was dissolved in 100 mmol/L potassium phosphate buffer, pH 6.1, 0.1% ascorbic acid.

Microtiter plate preparation: A glycerol stock of Lactobacillus rhamnosus ATCC 7469 was inoculated into 10 ml of Lactobacillus medium and incubated. The culture was grown to the logarithmic phase, and cells were collected by centrifugation (2500 G×5 minutes). The cell pellet was washed three times in 0.85% NaCl solution, suspended in 10 ml storage medium, and diluted 1:10 in the assay medium containing 200 mM/L trehalose. The dilution was adjusted so that 1 mi contained 10⁷ viable bacteria. 3 μl of the bacterial suspension was added to the bottom of each well of the microtiter plate, snap-frozen at −80° C. in the freezer and lyophilized by applying vacuum. Thus, each microtiter plate well contained exactly 3×10⁴ viable Lactobacillus rhamnosus germs of the same growth stage, enclosed in a trehalose/sugar/salt pellet that adhered to the bottom of the well. The stickiness of the pellet was further increased by adding small amounts of sucrose and dextrose to the freezing solution. The plates were packed sterile and light-tight with desiccant (Sica). Microtiter plates prepared in this manner are stable at room temperature for extended periods without loss of microbial viability.

Lactobacillus rhamnosus prefers an assay medium having pH 5 to 7. It is preferably to buffer the system at about pH 6 during cultivation to keep the growth of the Lactobacillus constant even in wells containing a high concentration of folic acid. Metabolism of folate results in lactic acid, the acidity of which may have an inhibitory effect on the growth rate at higher concentrations. This can be achieved by using an assay medium (ASYMED) buffered with 50 mM KHPO₄/KH₂PO₄ buffer, pH 6.1.

Cell lysis: 4.5 mL lysis buffer (HSOL: 0.1% ascorbic acid/ascorbate, pH 4.2) is combined with permeabilizing surfactant (PAF—0.1 g/l sapogenins in the final dilution). 25 μl whole blood sample was mixed with 225 μl HSOL/PAF (1:10) and incubated at 37° C. for 30 minutes. In this step, the ascorbic acid/ascorbate induces lysis of all blood cells, mainly red blood cells (RBC), and the γ-polyglutamates of folic acid (pteroyl polyglutamate—PteGluₙ) are released into the reaction solution and do not precipitate. Therefore, the solubilized pteroyl polyglutamates can be enzymatically hydrolyzed by the γ-glutamyl hydrolase released from the lysosomes of other blood cells, such as leukocytes and granulocytes. In this step, γ-glutamyl hydrolase can be added to the lysis buffer and the incubation time can be increased, but the γ-glutamyl hydrolase inherent in the lysosomes of whole blood cells is generally sufficient to lyse all pteroyl-polyglutamates, provided that the pH is not below pH 4.2, which is the case when unbuffered 0.1% ascorbic acid is used for cell lysis. The γ-glutamyl hydrolase is not active when the reaction buffer has such a low pH (pH 2.5 in the case of an unbuffered 0.1% ascorbic acid solution).

The higher pH is important during cell lysis because the acidic pteroyl-polyglutamates can stick to proteins and aggregate if the pH is too low. If the pH is too high or higher than pH 4.2, cell lysis may not be complete. Therefore, sapogenins are preferably added to ensure complete cell lysis and permeabilization of these cell compartments to glutamase conjugase (γ-glutamyl hydrolase). During development, additional processing steps were tested, and saponin was found to be optimal in combination with ascorbic acid/ascorbate buffer.

Incubation is followed by another sample dilution of 1:75, and the samples and CTRL/STD are added to the microbiological assay in a folate-deficient medium and incubated for 48 h. The microbial assay itself is performed as described previously.

The kit contains the following components:
Microtiter plate, each well pre-inoculated with 10⁴ cells of Lactobacillus rhamnosus;
SOL sample handling buffer 5×4.5 ml.
PAF Buffer folic acid in whole blood, lyophilized.
5×DIL water 4×30 ml.
ASYMED Folic acid assay medium 4×.
STD Folic acid standard, lyophilized 4×.
FOL Masking foil 4×.
FRA Spare frame for repositioning the microtiter strips 1×.
ASYBUF Folic acid medium treatment buffer 4×1.5 ml.
CTRL 1 Folic acid control 1, lyophilized 4×.
CTRL 2 Folic acid control 2, lyophilized 4×.

### Example 2 Comparison of Samples According to Invention with Samples Prepared According to the Method Published by Molloy et al

Preparation of whole-blood samples for erythrocyte folate analysis according to Molloy et al: The whole blood sample obtained from the individual was thoroughly mixed in a 1.5-ml Eppendorf tube and 100 μl thereof was added to 900 μl of 1% ascorbic acid solution, freshly prepared by adding 1 g ascorbic acid (not sodium ascorbate) to 100 ml of distilled water. The pH was not adjusted, resulting in a lysed mixture with a pH of approximately 2.3. The lysed mixture was then allowed to stand at room temperature for at least 30 to 40 minutes prior to the folate assay to allow serum conjugase to convert folate polyglutamates released from the erythrocytes to the assayable monoglutamate forms. The erythrocyte/ascorbic acid lysate was then stored in the freezer at below −20 degrees Celsius until assayed.

Preparation of samples according to the invention. In parallel, 25 μl of the mixed whole blood sample was mixed with 225 μl of 0.1% ascorbic acid/ascorbate, adjusted at pH 4.2 (HSOL) containing 0.1 g/l sapogenins (PAF) to a final dilution of 1:10 and incubated at 37° C. for 30 minutes. The erythrocyte/ascorbic acid lysate was then also stored in the freezer at below −20 degrees Celsius until assayed.

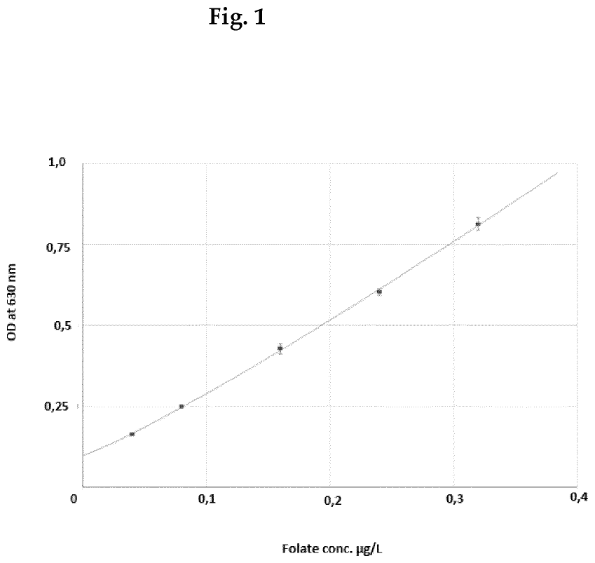

The results of comparative measurements from ten individuals are shown in Table I below. FIG. 1 shows the standard calibration curve (OD at 630 nm) used for the comparative measurements (in duplicates).

TABLE I

| | Folate status in | Folate status in | Deviation of measured | |
|---|---|---|---|---|
| | sample acc. to | sample acc. to | folate status absolute | |
| Sample No. | example I | Molloy et al. | proportional | |
| 1 | 79.44 | 65.25 | 14.19 | 17.9% |
| 2 | 60.74 | 48.64 | 12.10 | 19.9% |
| 3 | 62.45 | 46.79 | 15.66 | 25.1% |
| 4 | 48.07 | 35.61 | 12.46 | 25.9% |
| 5 | 62.84 | 61.65 | 1.19 | 1.9% |
| 6 | 44.55 | 28.36 | 16.19 | 36.3% |
| 7 | 71.88 | 51.44 | 20.44 | 28.4% |
| 8 | 59.23 | 54.99 | 4.25 | 7.2% |
| 9 | 54.46 | 34.52 | 19.94 | 36.6% |
| 10 | 51.93 | 32.43 | 19.50 | 37.6% |

As shown in Table I, the sample preparations according to the method of the invention result in tests in which the lysis of the cells (erythrocytes) is complete and in which the released folate polyglutamates are all enzymatically hydrolyzed to assayable forms. The high variability of the deviation of the folate status also indicates that the degree of polymerization within the folate polyglutamates was not constant among the tested individuals or followed a rule but can be taken as characteristic of the physiological state and folate supply of an individual. Although we do not wish to be bound by any theory, we believe that the low pH of the mixture, approximately 2.3 for samples prepared according to Molloy et al., causes the folate polyglutamate forms to become entrapped or aggregated with cell membranes so that they are only partially hydrolyzed by serum conjugase (=γ-glutamyl hydrolase). The inventors have found that complete enzymatic degradation of the folate polyglutamate forms can only be achieved in a buffer with a pH above 4.2, which pH shift is necessary because of the individually different degrees of glutamate polymerization. The low pH in the lysed cell mixture according to Molloy et al. and as used by the prior art methods therefore inevitably leads to an insufficient recovery of folate and an incorrect assessment of the folate status of a person. The influence of pH and saponin on cell lysis of whole blood in the context of erythrocyte folate analysis was previously studied and reported by Wright A J A et al in Erythrocyte Folate Analysis: Saponin Added During Lysis of Whole Blood Can Increase Apparent Folate Concentrations, Depending on Hemolysate pH, Clinical Chemistry (2000) 46:12 1978-1986. However, they have only examined a single blood sample and thus overlooked that the degree of polymerization within the folate polyglutamates is variable and also that the hydrolysis of the glutamates by the inherent conjugase is highly pH dependent and requires a pH shift. To the contrary, VWight AJA et al describe that a high pH of higher than 4.5 is combined with incomplete cell lysis and an incorrect low assessment of the folate status of a person.

1. A method for microbiological determination of folate and folic acid in a whole blood sample and assessment of the folate status of an individual, comprising the steps of:
preparing one or more culture vessels containing a predetermined number of viable cells of Lactobacillus rhamnosus for microbiological growth and metabolism determination;
obtaining a defined sample amount of whole blood from an individual whose folate status is to be determined;
adding to the sample of whole blood a predetermined amount of red blood cell lysis buffer to obtain lysis of red blood cells for a release of folate species;
adding an amount of γ-glutamyl hydrolase and/or a surfactant capable of lysosomal permeabilization to release lysosomal γ-glutamyl hydrolase from cells contained in the whole blood sample;
treating and incubating the lysed blood sample at pH 5.5 to 7 for a sufficient time to allow enzymatic hydrolysis of the γ-glutamyl chains from folypolyglutamate species, folytetraglutamates, folypentaglutamates, and folyhexaglutamates;
performing a microbiological assay to compare growth and metabolism in the absence or presence of varying amounts of the treated whole blood sample and/or folate calibrator to assess the individual's folate status against folate references.

2. The method of claim 1, wherein the red blood cell lysis buffer contains ascorbate/ascorbic acid, at pH 4.2 to 5.0, and a detergent for permeabilization of lysosomes.

3. The method of claim 1, wherein the surfactant for lysosomal permeabilization is selected from the group consisting of sapogenins, steroidal sapogenins, saponins, triterpene glycosides, terpenoids, alkylphenol ethoxylates, (Triton® X-100), nonylphenol ethoxylates, and octylphenol ethyxylates.

4. The method of claim 1, wherein the enzymatic hydrolysis of γ-glutamyl chains from folypolyglutamate species is carried out in a phosphate buffer of pH 5.5 to 6.5 at ambient temperature to 37 degrees Celsius for 10 to 30 minutes.

5. The method of claim 1, wherein Lactobacillus rhamnosus is grown in an assay medium buffered at pH 6 to counteract the inhibitory effects of lactic acid production.

6. The method of claim 1, wherein the Lactobacillus is Chloramphenicol-resistant Lactobacillus rhamnosus ATCC 7469.

7. The method of claim 1, wherein a microtiter plate is used as the culturing vessel.

8. The method of claim 1, wherein a test kit is used for assessing the folate status of an individual by microbiological assay of folate and/or folic acid in a whole blood sample, wherein the test kit comprises:
a microtiter plate which cavities each contain a predetermined number of viable cells of Lactobacillus rhamnosus, that have been rendered durable for dry storage at ambient temperature by shock-freezing and freeze-drying;
an ascorbic acid/ascorbate buffer system, pH 4.0 to 4.5.
